(19) United States
(12) Patent Application Publication
(10) Pub. No.: US 2024/0374120 A1
(43) Pub. Date: Nov. 14, 2024

(54) A CONTROL VALVE FOR AN ENDOSCOPE

(71) Applicant: GA Health Medical Devices Limited, Mullingar, Co. Westmeath (IE)

(72) Inventor: Steven Chau, ShekMun, Shatin (CN)

(21) Appl. No.: 18/683,190

(22) PCT Filed: Aug. 18, 2022
(86) PCT No.: PCT/EP2022/073135

§ 371 Date: Feb. 12, 2024

(30) Foreign Application Priority Data
Aug. 18, 2021 (GB) 2111843.5

(51) Int. Cl. A61B 1/00; A61B 1/015

(52) U.S. Cl. CPC A61B 1/00068; A61B 1/0011; A61B 1/015

(57) ABSTRACT

A control valve assembly (1, 50) for an endoscope comprising a stem (2) having a longitudinal axis, a first opening (10) disposed transverse to the longitudinal axis, the first opening for allowing passage of air and/or fluid, a cap mountable to the top of the stem, a spring stanchion (3) comprising an opening configured to receive the stem and allow movement of the stem in an upward and downward position relative to the spring stanchion, and a spring (4) configured to contact the spring stanchion (3) and the cap and bias the stem upwardly with respect to the spring stanchion. The cap comprises first cap part (5, 51) having an opening to receive the stem and configured for coupling to the top of the stem (2) for movement with the stem, and a second cap part (6, 52) having an opening to receive the stem and configured to contact the spring and allow movement of the second cap part in an upward and downward position relative to the spring stanchion. The first cap part and second cap part are not coupled together and are configured to cooperate during upward and downward movement of the stem.

### FIELD OF THE INVENTION

The present invention relates to a control valve for an endoscope, and a method of manufacturing a control valve for an endoscope.

### BACKGROUND TO THE INVENTION

Endoscopes are well-known in the art and are commonly used for numerous medical procedures. A control section of an endoscope may include a suction cylinder, air/water cylinder, and the like. Valves may be inserted into these cylinders to control various functions of the endoscope. The control valves may be reusable or disposable. A control valve assembly for an endoscope according to the preamble of claim 1 is described in WO2012/075116. In this control valve assembly, the top of the stem comprises an integrally formed cap and the spring contacts an underside of the cap. The boot is over-moulded on the spring stanchion. An alternative control valve assembly is sold by ANDORATE under the product code GAR004C (air/water valve) and GAR004 (suction valve). In this assembly, an underside of the cap has a threaded socket configured to engage a threaded top of the stem and an annular recess dimensioned to receive and abut a top of the spring. In practice, the cap has a tendency to unscrew from the stem during use, and adhesive is generally applied to further secure the cap to the stem. This complicates the assembly of the control valve making it unsuitable for high-throughput and automated assembly.

It is an objective of the invention to overcome at least one of the above-referenced problems.

### SUMMARY OF THE INVENTION

The objective is met by the provision of a cap is two parts, namely, a first cap part is configured for mounting on the stem and to couple (e.g. snap-fit) to the top of the stem, and a second cap part is configured for mounting on the stem in contact with the spring for movement along the stem. Upon assembly, the second cap part is urged into cooperation with the first cap part and the two parts therefore cooperate during upward and downward movement of the stem by the user. As the part of the cap that engages the stem and the part of the cap that contacts the spring are decoupled, this facilitates manufacture and provides for a more stable connection. In addition, this configuration is more suited to automated assembly as it does not require the threaded engagement of parts or the use of adhesive.

In a first aspect, the invention provides a control valve assembly for a medical device such as an endoscope comprising:
a stem having a longitudinal axis, a first opening disposed transverse to the longitudinal axis, the first opening for allowing passage of air and/or fluid;
a cap mountable to the top of the stem;
a spring stanchion comprising an opening configured to receive the stem and allow movement of the stem in an upward and downward position relative to the spring stanchion; and
a spring configured to contact the spring stanchion and the cap and bias the stem upwardly with respect to the spring stanchion,
   characterised in that the cap comprises:
   a first cap part having an opening to receive the stem and configured for coupling to the top of the stem for movement with the stem; and
   a second cap part having an opening to receive the stem and configured to contact the spring and allow movement of the second cap part in an upward and downward position relative to the spring stanchion, wherein the first cap part and second cap part are configured to cooperate during upward and downward movement of the stem.

The control valve assembly may be a suction valve assembly or an air/water valve assembly.

In any embodiment, the first cap part is configured for snap fit attachment to the top of the stem.

In any embodiment, the first cap part comprises a radially inward flange configured for snap-fit coupling to a corresponding recess in the top of the stem.

In any embodiment, the radially inward flange comprises a first flange part and a second flange part. The first and second flange parts may each be partly annular.

In any embodiment, the recess in the top of the stem may be a fully or partially annular recess.

In any embodiment, the second cap part comprises a cylindrical element and the first cap part comprises a cylindrical recess dimensioned to receive the cylindrical element in an abutting relationship when the first and second cap parts cooperate together.

In any embodiment, the radially inward flange is disposed radially inwardly of the cylindrical recess.

In any embodiment, the first cap part comprises a cylindrical element that is not fully annular (e.g. an incomplete ring) and is resiliently deformable from an un-tensioned expanded configuration suitable for receiving the stem and a contracted tensioned configuration in which the cylindrical element snap fits to the stem.

In any embodiment, the cylindrical element has an exterior sidewall that tapers inwardly towards a base thereof.

In any embodiment, the cylindrical element comprises a radially inward partially annular flange configured for snap-fit coupling to a corresponding annular recess in the top of the stem.

In any embodiment, the second cap part comprises a cylindrical recess dimensioned to contact the spring (e.g. a top of the spring) and an annular shoulder disposed radially inwardly of the cylindrical recess configured to cooperate with a the cylindrical element, generally a base of the cylindrical element.

In any embodiment, the spring stanchion comprises at least one recess or projection configured to cooperate with a corresponding recess or projection on the step, the recess and projection being configured to align the stem and stanchion during assembly and limit the upward and downward movement of the stem relative to the stanchion during use.

In any embodiment, the stanchion comprises projections (e.g. tabs) that project into the opening of the stanchion and an outer surface of the stem comprises corresponding elongated recesses configured to slidably receive the tabs when the stanchion is mounted on the stem.

In any embodiment, the opening in the spring stanchion is disposed in a centre of the spring stanchion and the spring stanchion comprises a ledge configured to receive a lower end of the spring.

In any embodiment, the suction valve assembly includes a resiliently deformable boot configured for mounting around the stanchion. The boot allows the assembled control valve to be sealingly mounted in a control cylinder of a medical device.

In any embodiment, the stem has a diameter that is concentric to the diameter of the boot to assure an airtight seal within a control cylinder of a medical device.

In any embodiment, the stem, and/or spring stanchion comprise disposable thermoplastic material.

In any embodiment, the medical device is an endoscope and the control valve is a suction valve or an air/water valve (and the cylinder is generally a suction cylinder or an air water cylinder). When the control valve is a suction valve, the stem generally includes a lumen providing fluidic communication from the cylinder of an endoscope to the first transverse aperture.

In any embodiment, the suction valve assembly is disposable.

In a second aspect, the invention provides a medical device comprising a control valve assembly according to the invention.

In any embodiment, the control valve assembly is an endoscope.

In any embodiment, the control valve is a suction valve or an air/water valve (and the cylinder is generally a suction cylinder or an air water cylinder). When the control valve is a suction valve, the stem generally includes a lumen providing fluidic communication from the cylinder of an endoscope to the first transverse aperture.

In another aspect, there is provided a method for manufacturing a control valve (typically a disposable control valve) comprising:
providing (e.g. by molding) a stem;
providing (e.g. by molding) a spring stanchion;
placing a top end of the stem through a stem opening in the spring stanchion;
placing a top end of the stem though a centre of the spring and bringing a bottom end of the spring into contact with the spring stanchion;
placing a top end of the spring into contact with a second cap part and applying downward pressure to the second cap part against the spring bias so that the top end of the stem projects through an opening in the second cap part;
while downward pressure is maintained on the second cap part, placing the top end of the stem through an opening in a first cap part until the first cap part couples (e.g. snap-fits) to the stem;
and releasing the downward pressure on the second cap part to allow the second cap part be pushed upwardly by the spring into cooperation with the first cap part.

In any embodiment the method includes a step of placing a boot around the spring stanchion.

Other aspects and preferred embodiments of the invention are defined and described in the other claims set out below.

### BRIEF DESCRIPTION OF THE FIGURES

FIGS. 1 to 9 illustrate a suction valve assembly according to the invention in which:

FIG. 1 is an exploded view of part of a suction valve assembly for an endoscope.

FIG. 2 is a view of the parts of the assembly of FIG. 1 in an assembled form.

FIG. 3 is a view of the first cap part before coupling to the stem of the assembly of FIG. 2.

FIG. 4 is a sectional view of the assembly of FIG. 3.

FIG. 5 is a perspective view of the suction valve assembly prior to attachment of the silicone cover.

FIG. 6 is a perspective sectional view of the assembly of FIG. 5.

FIG. 7 is a top plan view of the assembly of FIG. 5 prior to the silicone cap being attached.

FIG. 8 is a perspective partly exploded view showing a boot prior to attachment to the spring stanchion of the suction valve assembly.

FIG. 9 is a perspective sectional view showing the suction valve assembly fully assembled.

FIGS. 10 to 20 illustrate an air/water valve for an endoscope, according to the invention, in which

FIG. 10 illustrates the assembly of a stem for the air/water (A/W) valve assembly.

FIG. 11 illustrates the assembly of the stem, spring, stanchion and boot parts of the air/water valve assembly for an endoscope.

FIG. 12 illustrates the stem, spring, stanchion and boot parts in an assembled form.

FIG. 13 illustrates the attachment of the second cap part to the assembly of FIG. 12.

FIG. 14 illustrates the first cap part in an un-tensioned expanded configuration and

FIG. 15 illustrates the first cap part in a tensioned contracted configuration

FIG. 16 illustrates the first cap part in the expanded configuration being axially mounted over the top of the stem while the second cap part is held down against the spring bias.

FIG. 17 illustrates the first cap part snap-fitted to the stem while the second cap part is held down against the spring bias.

FIG. 18 illustrates the second cap part after being released and spring-biased upwardly into cooperation with the first cap part.

FIG. 19 is a side elevational view of the air/water valve assembly in a fully assembled form.

FIG. 20 is a sectional view of the assembled air/water valve of FIG. 19.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

“Control valve” refers to a valve that is used with a medical device like an endoscope and is used to control the flow of fluid into or out of the endoscope. The control valve may be a suction valve or an air/water (or A/W) valve. The valve generally has a spring stanchion with a central opening, a stem configured for mounting through the opening of a spring stanchion, a cap at the top of the stem (e.g. aq button that is depressed by a user), and a spring mounted between the spring stanchion and the cap of the stem. The stem generally has a transverse through hole, and may also have an axial lumen to provide fluid communication between the cylinder and transverse through hold. The spring biases the stem into an upward position in which the valve is closed. Depressing the stem into the cylinder results in the transverse through hole being aligning with an outlet to open the valve. In the present invention, the cap is provided in two parts that cooperate together during use, a first part that couples (e.g. snap fits) to the stem and a second part that contacts the spring. Thus, when assembled, the second part is biased by the spring into an abutting relationship with the first part.

“Stem” is generally an elongated post mountable in a cylinder of a medical device and upwardly and downwardly movable to open and close the control valve. The stem generally includes a transverse through hole that in use can be aligned with a fluidic outlet in the cylinder to allow fluid flow between the cylinder and the fluidic outlet. The low part of the stem may include a lumen with an opening at the bottom of the stem and a further opening into the transverse through hole. The stem may be cylindrical or may have one or more annular indents configured to receive sealing O-rings.

“Spring stanchion” is a generally cylindrical body with a central opening configured to receive the stem. The stanchion may include formations (e.g. tabs) that project radially inwardly into the opening and are configured to engage corresponding elongated recesses on an outer wall of the stem. The purpose of the formations and recesses is to guide the insertion of the stem into the opening of the stanchion, and also to limit the movement of the stem relative to the stanchion (e.g. to limit how far the stem can be moved upwardly relative to the stanchion). The stanchion also includes a ledge, generally an annular ledge, disposed around the opening that is configured to receive the base of the spring.

“Spring” is generally a helical spring configured to embrace the stem. However, it will be appreciated that other forms of springs (e.g. resiliently deformable members) may be employed to contact the stanchion and second cap part and urge the second cap part into cooperation with the first cap part. For example a rubber gasket that is resiliently deformable in a longitudinal direction may be employed

“Boot” refers to a covering for the stanchion that assists the sealing engagement of the stanchion in the cylinder of the medical device. It is generally formed from a resiliently deformable material that tightly adheres to a sidewall of the stanchion. The boot may be longitudinally oversized with respect of the stanchion (e.g. it extends axially proud of one or both ends of the stanchion.

“Cap” refers to the button at the top of the stem that is pressed by a user to actuate the valve. The cap of the invention is a two-part cap comprising two parts that cooperate together during use, typically a first part that couples (e.g. snap fits) to the stem and a second part that contacts the spring. Thus, when assembled, the second part is biased by the spring into an abutting relationship with the first part.

“First cap part” refers to the part of the cap that engages the stem and cooperates with the second cap part. It is generally a partially or fully annular body with a central opening to receive the stem. It may have an radially inwardly projecting flange that defines the opening. The flange may be fully annular, or may be provided by two flange parts, each of which may be partially annular. The flange is deformable to allow the first cap part be mounted on the top of the stem and allow the flange spring back to engage a partially or fully annular recess on the top of the stem. The top of the stem may be conical or frustoconical to urge the flange away from the opening when the first cap part is mounted on the stem. The first cap part may comprise a cylindrical element that is not fully annular and is resiliently deformable from an un-tensioned expanded configuration suitable for receiving the stem and a contracted tensioned configuration in which the cylindrical element snap fits to the stem.

“Second cap part” refers to the part of the cap that is mounted on the stem for axial movement upwards and downwards on the stem and is configured to contact the top of the spring for urging the second cap part into cooperation with the first cap part. The second cap part is usually a cylindrical body with a central opening configured to receive the stem and comprises a ledge configured to contact the top of the spring. The ledge may comprise an annular recess configured to receive the spring. The second cap part may be a cylindrical body with a radially outward flange disposed at the top of the cylindrical body. The cylindrical body may have a radially inward projecting flange defining the opening and an annual recess for receiving the top of the spring disposed radially outwardly of the of the radially inward flange.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

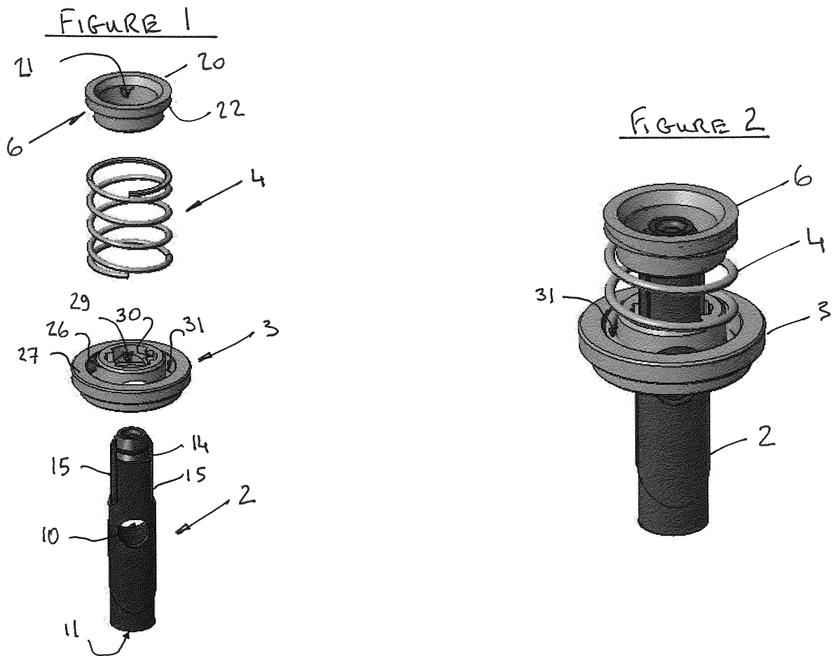

Referring to FIGS. 1 to 9, a suction valve assembly according to the invention is illustrated and indicated generally by reference numeral 1. The suction valve is for use with a suction cylinder of an endoscope and can be actuated by a user by depressing the valve to apply a negative pressure to the suction cylinder of an endoscope to remove fluid from the working conduits of the endoscope. Depressing the valve against the spring bias allows a user during use of the endoscope apply suction to the conduits of the endoscope to remove fluid or debris which may be obscuring the view. Due to the spring bias, once the user takes their find off the valve, the suction is stopped.

The suction valve assembly 1 comprises a stem 2, spring stanchion 3 (referred to hereafter as “stanchion”), spring 4, first cap part 5, and second cap part 6, which are assembled together in a specific sequence to form the suction valve assembly 1.

The stem 2 is an elongated cylinder with a top part 12, bottom part 13, an axial through hole 11, and transverse through hole 10 disposed intermediate the top part 12 and bottom part 13. The top part 12 of the stem has an annular circumferential recess 14 and longitudinal recesses 15A on opposed sides of the stem 1 that define tabs 15B. The purpose of the circumferential recess 14 is to couple with the first cap part 5 as is described in more detail below. Below. The purpose of the longitudinal recesses 15A and tabs 15B is to guide the mounting of the stanchion 3 on the stem and to limit the upward and downward movement of the stem relative to the stanchion (which in use is fixed in position in the endoscope suction cylinder). The uppermost part of the stem 2 is chamfered inwardly to facilitate mounting of the first cap part 5 on the top of the stem 2.

Referring to FIGS. 1 and 2, the second cap part 6 is described in more detail and comprises a cylindrical body 20 with a central opening 21 dimensioned to receive and slide freely up and down the stem, and a flange 22 that extends radially outwardly of the top of the body 20. Referring to FIG. 4 it can be seen that the flange 23 is dimensioned to provide a circumferential recess to receive the first turn of the spring 4.

The stanchion 3 comprises a cylindrical body 25 with an inner annular wall 26, outer annular wall 27 and base wall 28 connecting the bottom of the inner and outer walls. The inner wall 26 defines a central opening 29 and comprises opposed sidewall formations 30 that project partially into the opening and in use engage the longitudinal recesses 15A and tabs 15B of the stem to guide the mounting of the stanchion on the stem and limit the upward movement of the stem on the stanchion. The inner and outer annular walls define a recess 31 dimensioned to receive the bottom of the spring 4.

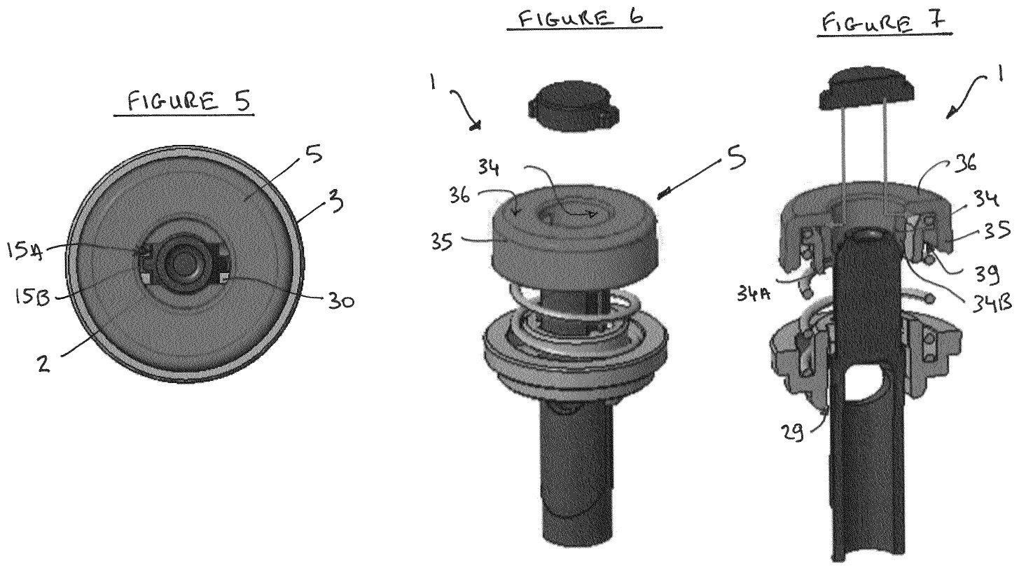

Referring to FIGS. 5 and 7, the first cap part 5 is described in then more detail and comprises a cylindrical body 33 defined by an inner partly annular sidewall 34, outer annular sidewall 35 and bridging wall 36 connecting the top of the inner and outer sidewalls and forming an annular recess 39 dimensioned to receive the top of the second cap part 6. The inner party annular sidewall 34 comprises a first inner sidewall part 34A and opposed second inner sidewall part 34B, each having a radially inwardly projecting flange section 37 disposed at a base thereof. The flange sections are resiliently deformable in a radially outward direction to receive the top of the stem and snap-fit closed around the annular circumferential recess 14 of the stem to couple the first cap part 5 to the stem 2.

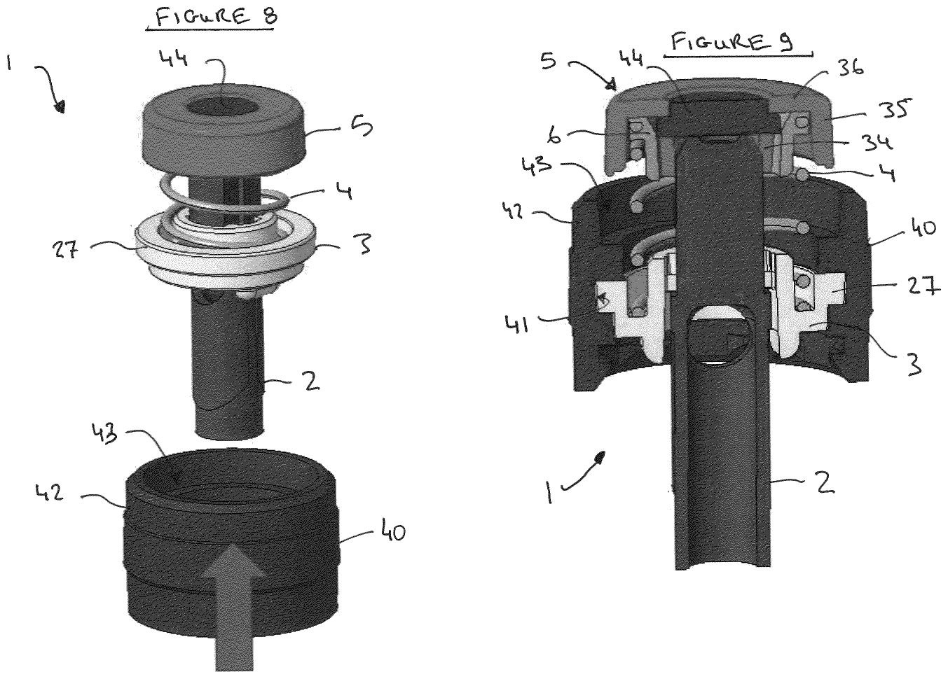

Referring to FIGS. 8 and 9, the assembly 1 comprises a boot 40 dimensioned to rightly embrace the stanchion 3 and having an inner annular recess 41 configured to mate with an outer sidewall 27 of the stanchion 3. The boot is longitudinally oversized with respect to the stanchion and has an upper part 42 providing an annular recess 43 dimensioned to receive the first and second cap parts in a nested arrangement such that when the stem is fully depressed the top of the second cap part extends proud of the top of the boot. This provides tactile feedback to the user who will know that the valve has been fully actuated. A silicone cap 44 is then mounted in the top opening of the second cap part to form a fluidically tight seal.

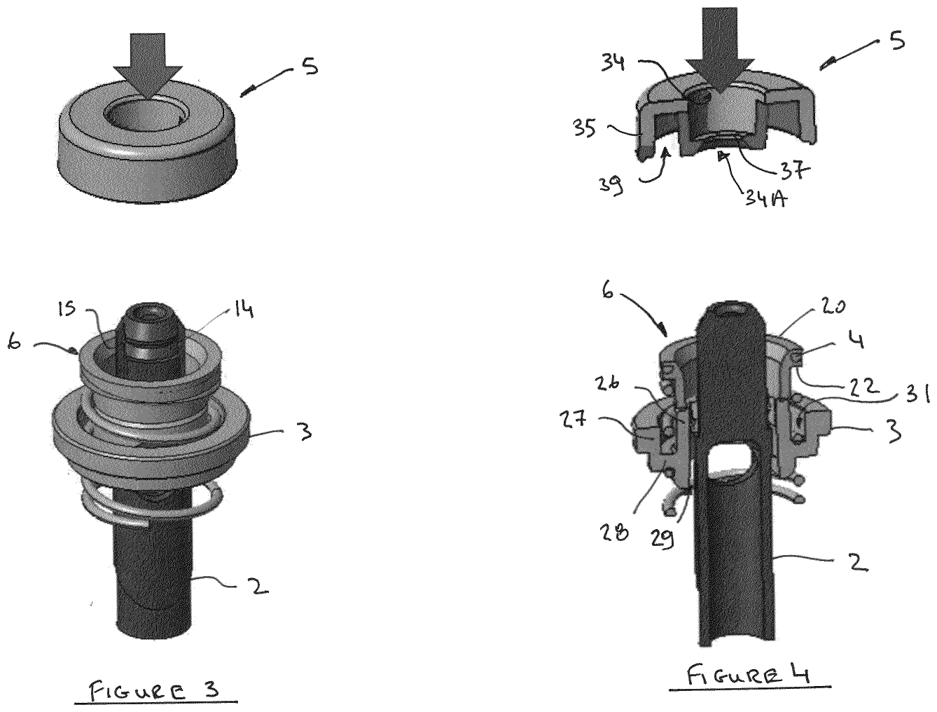

To assemble the suction valve, and referring initially to FIGS. 1 to 4, the stanchion 3 is positioned on top of the stem 2 such that the opposed formations 30 on the stanchion and elongated recesses 15A and tabs 15B on the stem are aligned and lowered as far as the recesses 15A allow. The spring 4 is then mounted on the top of the stem 2 and the second cap part 6 is then mounted on the top of the spring 4 and pushed down around the top of the stem to compress the spring and held in a depressed position shown in FIG. 3 so that the top of the stem 2 is exposed proud of a top of the second cap part 6. The second cap part can be held in the depressed position using a hand or a suitable tool such as a small spanner.

Referring to FIGS. 5 and 6, the first cap part 5 is then mounted on the top of the stem 2 with the top of the second cap part 5 being received in the annular recess 39 of the first cap part 5. The first cap part 5 is then pushed downwardly until the opposed sidewall flange parts 34A and 34B snap-sit engage the circumferential recess 14 on the top of the stem, locking the first cap part 5 to the stem 2. The boot is then mounted over the bottom part of the stem 2 and placed around the stanchion 3 such that the inner annular recess 41 mates with an outer sidewall 27 of the stanchion 3 securing the boot 40 in position on the stanchion 3. The retaining force on the second cap part 6 is then released, and the second cap part 6 is forced upwardly relative to the stem 2 into cooperation with the first cap part 5. Finally, the silicone cap 44 is mounted in the top opening of the first cap part to form a fluidically tight seal. The suction valve is then ready to be mounted in a suction cylinder of an endoscope, there the boot is configured to position and couple the valve to the suction cylinder.

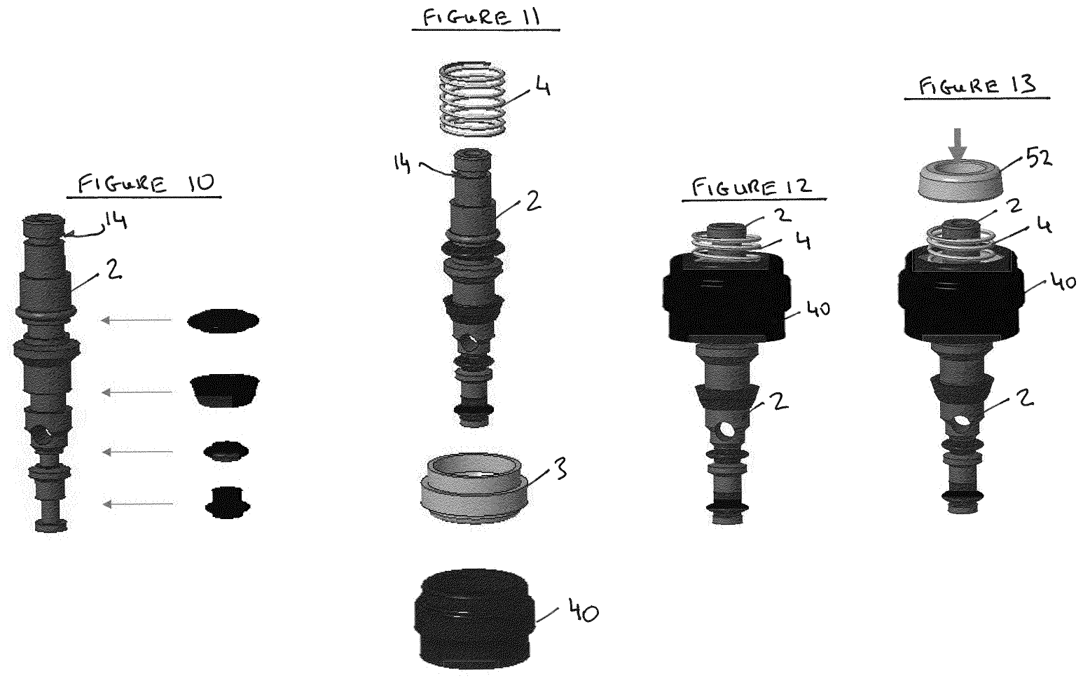

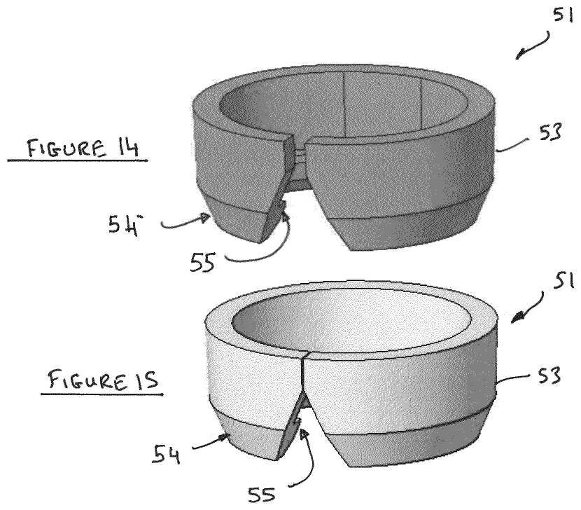

Referring to FIGS. 10 to 20, an alternative control valve assembly of the invention is described, in this case an air/water (A/W) valve, indicated generally by the reference numeral 50, and in which parts described with reference to the previous embodiment are assigned the same reference numerals. In this embodiment, the first cap part 51 comprises a cylindrical element 53 that is not fully annular and is resiliently deformable from a tensioned expanded configuration suitable for receiving the stem (FIG. 14) and a contracted un-tensioned configuration in which the cylindrical element snap fits to the annular recess 14 at the top of the stem 2 (FIG. 15). Referring to FIGS. 14, 15 and 19, the cylindrical element 53 has an exterior sidewall with an inwardly tapering base 54 and a radially inward flange 55 dimensioned to snap fit engage with the annular recess 15 on the stem.

Referring to FIGS. 19, the second cap part 52 comprises a cylindrical body 57 defined by an inner annular sidewall 58, outer annular sidewall 59 and bridging wall 60 connecting the top of the inner and outer sidewalls and forming an annular recess 61 dimensioned to receive the top of the spring 4. The inner party annular sidewall 58 has a radially inward annular flange 62 that defines a stem-receiving opening that is slightly wider than the stem and allows sliding movement of the second cap part on the stem. When mounted on the stem, the annular flange 62 provides a circumferential recess 67 (FIG. 17) dimensioned to receive the first cap part 51 when the second cap part 52 is urged into cooperation with the first cap part 51.

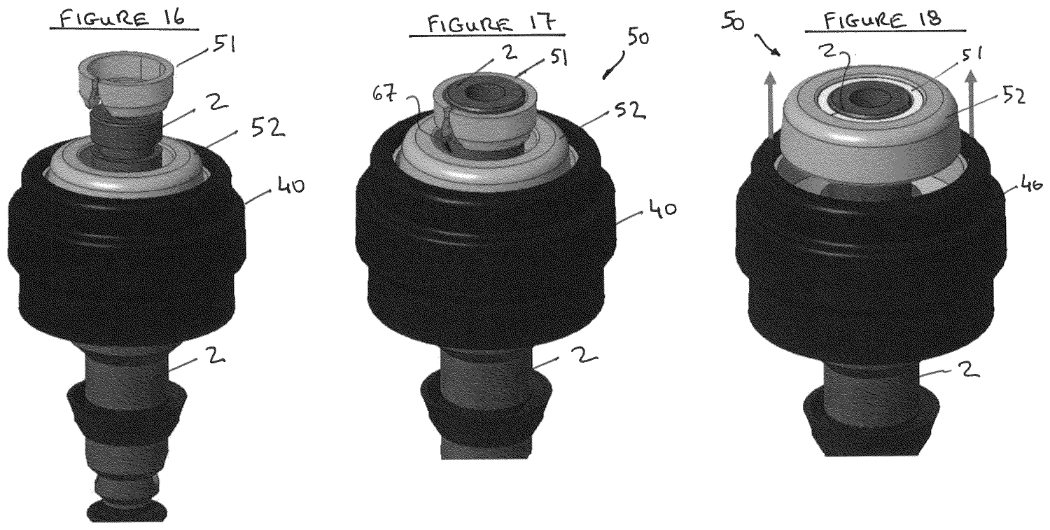

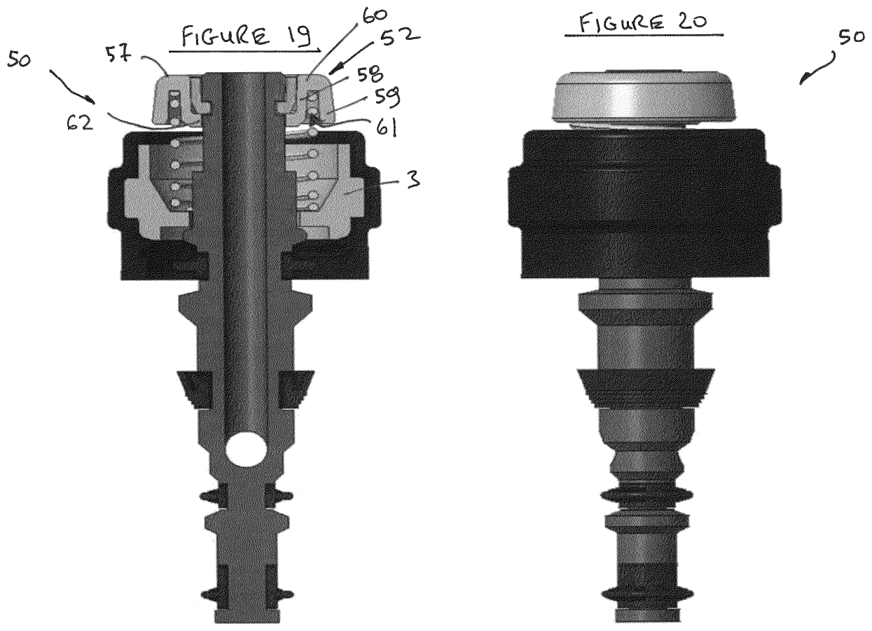

The assembly of this embodiment of the valve of the invention is substantially the same as that described with reference to the embodiment of FIGS. 1 to 9, with the stanchion being mounted on the stem, the spring being mounted on the stem with the spring base supported on a ledge of the stanchion, the second cap part being mounted on the top of the stem and pushed downwardly so that the second cap part engages the top of the spring and compresses the spring (See FIGS. 10-14). The first cap part is then mounted on the exposed top of the stem and forced downwardly to expand the cylindrical element allowing it embrace the top of the stem and then snap-fit into the annular recess on the top of the stem (FIGS. 16-18). The retaining pressure on the second cap part 52 is then released and the second cap part moves upwardly relative to the spring to abut and fully embrace the first cap part (FIG. 18). In this embodiment, the boot is applied to the stanchion before the stanchion is mounted on the stem, and the boot is longitudinally oversized such that the top of the boot receives most of the second cap part when the valve is actuated and the first and second cap parts depressed by a user. FIGS. 19 and 20 show the air/water valve fully assembled, with the first cap part 51 and second cap part 52 cooperating and exposed above the boot 40.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A control valve assembly for an endoscope comprising:
a stem having a longitudinal axis, a first opening disposed transverse to the longitudinal axis, the first opening for allowing passage of air and/or fluid;
a cap mountable to the top of the stem;
a spring stanchion comprising an opening configured to receive the stem and allow movement of the stem in an upward and downward position relative to the spring stanchion; and
a spring configured to contact the spring stanchion and the cap and bias the stem upwardly with respect to the spring stanchion, characterised in that the cap comprises:
a first cap part having an opening to receive the stem and configured for coupling to the top of the stem for movement with the stem; and
a second cap part having an opening to receive the stem and configured to contact the spring and allow movement of the second cap part in an upward and downward position relative to the spring stanchion, wherein the first cap part and second cap part are not coupled together and are configured to cooperate during upward and downward movement of the stem,
and wherein the first cap part is configured for snap fit attachment to the top of the stem.

2. The control valve assembly according to claim 1, selected from a suction cylinder or an air water cylinder of an endoscope.

3. The control valve assembly according to claim 1, in which the first cap part comprises a radially inward flange configured for snap-fit coupling to a corresponding recess in the top of the stem.

4. The control valve assembly according to claim 1, in which the first cap part comprises a radially inward flange configured for snap-fit coupling to a corresponding recess in the top of the stem, in which the radially inward flange comprises a first flange part and a second flange part.

5. The control valve assembly according to claim 1, in which the second cap part comprises a cylindrical element and the first cap part comprises a cylindrical recess dimensioned to receive the cylindrical element in an abutting relationship.

6. The control valve assembly according to claim 1, in which the first cap part comprises a radially inward flange configured for snap-fit coupling to a corresponding recess in the top of the stem, and in which the second cap part comprises a cylindrical element and the first cap part comprises a cylindrical recess dimensioned to receive the cylindrical element in an abutting relationship, in which the radially inward flange is disposed radially inwardly of the cylindrical recess.

7. The control valve assembly according to claim 1, in which the first cap part comprises a cylindrical element that is not fully annular and is resiliently deformable between an un-tensioned expanded configuration suitable for receiving the stem and a contracted tensioned configuration in which the cylindrical element snap fits to the stem.

8. The control valve assembly according to claim 1, in which the first cap part comprises a cylindrical element that is not fully annular and is resiliently deformable between an un-tensioned expanded configuration suitable for receiving the stem and a contracted tensioned configuration in which the cylindrical element snap fits to the stem, in which the cylindrical element has an exterior sidewall that tapers inwardly towards a base thereof.

9. The control valve assembly according to claim 1, in which the second cap part comprises a cylindrical element and the first cap part comprises a cylindrical recess dimensioned to receive the cylindrical element in an abutting relationship, in which the cylindrical element comprises a radially inward annular flange configured for snap-fit coupling to a corresponding annular recess in the top of the stem.

10. The control valve assembly according to claim 1, in which the second cap part comprises a cylindrical recess dimensioned to receive and contact the spring and an annular shoulder disposed radially inwardly of the cylindrical recess configured to cooperate with a base of the first cap part.

11. The control valve according to claim 1, in which the spring stanchion comprises at least one recess or projection configured to cooperate with a corresponding recess or projection on the stem, the recess and projection being configured to align the stem and stanchion during assembly and limit the upward and downward movement of the stem relative to the stanchion during use.

12. The control valve assembly according claim 1, wherein the opening in the spring stanchion is disposed in a centre of the spring stanchion and the spring stanchion comprises a ledge configured to receive a lower end of the spring.

13. The control valve assembly according claim 1, including a resiliently deformable boot configured for mounting around the stanchion.

14. The control valve assembly according to claim 1, including a resiliently deformable boot configured for mounting around the stanchion, wherein the stem has a diameter that is concentric to the diameter of the boot to assure an airtight seal within a control cylinder of a medical device.

15. The control valve assembly according claim 1, wherein the stem and spring stanchion comprise disposable thermoplastic material.

16. The control valve assembly according to claim 1, wherein the control valve is a suction valve or an air/water valve for an endoscope.

17. An endoscope comprising a control valve assembly according to claim 1.

18. A method for manufacturing a disposable suction valve comprising:
molding a stem;
molding a spring stanchion;
placing a top end of the stem through a stem opening in the spring stanchion;
placing a top end of the stem though a centre of the spring and bringing a bottom end of the spring into contact with the spring stanchion;
placing a top end of the spring into contact with a second cap part and applying downward pressure to the second cap part against the spring bias so that the top end of the stem projects through an opening in the second cap part;
while downward pressure is maintained on the second cap part, placing the top end of the stem through an opening in a first cap part until the first cap part snap-fits to the stem;
and releasing the downward pressure on the second cap part to allow the second cap part be pushed upwardly by the spring into cooperation with the first cap part.

19. The method of claim 18 further comprising placing a boot onto the spring stanchion.
